(19) United States
(12) Patent Application Publication
(10) Pub. No.: US 2012/0022474 A1
(43) Pub. Date: Jan. 26, 2012

(54) PLASTER WITH MEDICATION DISPENSER

(76) Inventors: Sharon Adoni, Reut (IL); Simi Adoni, Reut (IL)

(21) Appl. No.: 12/988,239

(22) PCT Filed: Apr. 16, 2009
(86) PCT No.: PCT/IL2009/000418

§ 371 Date: Oct. 15, 2010

Related U.S. Application Data
(60) Provisional application No. 61/071,195, filed on Apr. 17, 2008.

(51) Int. Cl. A61F 13/02; A61M 35/00

(52) U.S. Cl. 604/307

(57) ABSTRACT

An adhesive plaster (10; 110; 600; 800) provided with one or more rupturable capsules (18; 118; 618; 818) containing one or more kinds of medication.

### FIELD OF THE INVENTION

The present invention generally relates to medication delivery methods and more particularly to plasters.

### BACKGROUND OF THE INVENTION

Many different methods and products were developed and are available, for addressing the matter of skin wounds protection and healing. One of the most commonly used products is the adhesive plaster also known as “band-aid”. The plaster has two main parts, the pad that covers the wound and the adhesive material that sticks to the skin and holds the plaster in place in order to achieve an optimal contact of the administered medication with the wound. The conventional plaster, therefore facilitates the application of a medication to the wound for a relatively long duration of time and isolate the wound from external harming conditions and also creates a fairly sterile environment, thereby accelerating the healing of the wound and preventing it from becoming inflammatory.

Although the plaster is highly applicable and quite efficient, it still holds a few disadvantages. For example, since the adhesive plaster by itself does not hold any healing qualities, one always needs to have the medication nearby. In terms of hygiene, the need to apply the medication directly to the pad imposes that the pad is exposed for a relatively long time to the environment and to other infecting factors, such as unclean fingers, dirt and dust, that might cause the inflammation of the pad.

Also, since the medication may be applied to the pad only in a small, limited amount, one needs to replace the plaster every few hours. This frequent replacement might cause irritation of the skin adjacent to the wounded area by the adhesive material and it also exposes the wound to the environment.

Many attempts have been made to tackle part of the above listed problems. Hence, for example, in U.S. Pat. No. 3,297,032 there has been disclosed an adhesive bandage comprising an adhesive cover, an absorbent pad (smaller than the adhesive cover) and a protective layer attached to the adhesive cover and covering pad. The protective layer carried a rupturable container of medicament facing the pad. This method, however, does not completely protect the medicament from becoming contaminated when opened.

It is the object of the present invention to cope with one or more of these disadvantages and provide a more efficient method of delivering medication to a wounded skin, while still maintaining at least some advantages of the conventional plaster.

### SUMMARY OF THE INVENTION

According to the invention there is provided a plaster for treating a wounded area.

Preferably, the plaster comprises: an adhesive cover; a first pad of a liquid absorbent material for covering the wounded area; and at least one squeezable medication containing container. Preferably, the pad and the container are arranged such that squeezing the container releases the medication to the pad.

Optionally, the squeezable container is a capsule made of a material adapted to be ruptured under pressure applied thereagainst.

Optionally, the plaster comprises a second pad, between the first pad and the adhesive cover. In some embodiments, one or more of the medication container(s) are between the first and the second pad. In some embodiments, the container(s) have only a portion between the two pads. Optionally, this portion is a narrowing portion, allowing to release the medication when the container is squeezed, and preventing medication from being released when the container is not squeezed.

In some embodiments, squeezing the container releases the medication to the interface between the two pads.

In some embodiments, squeezing the container releases the medication to an interface between the first pad and the wounded area.

### BRIEF DESCRIPTION OF THE PREFERRED EMBODIMENTS

These and additional constructional features and advantages of the invention will become more readily understood in the light of the ensuing description of some preferred embodiments thereof, given by way of example only, with reference to the accompanying drawings, wherein:

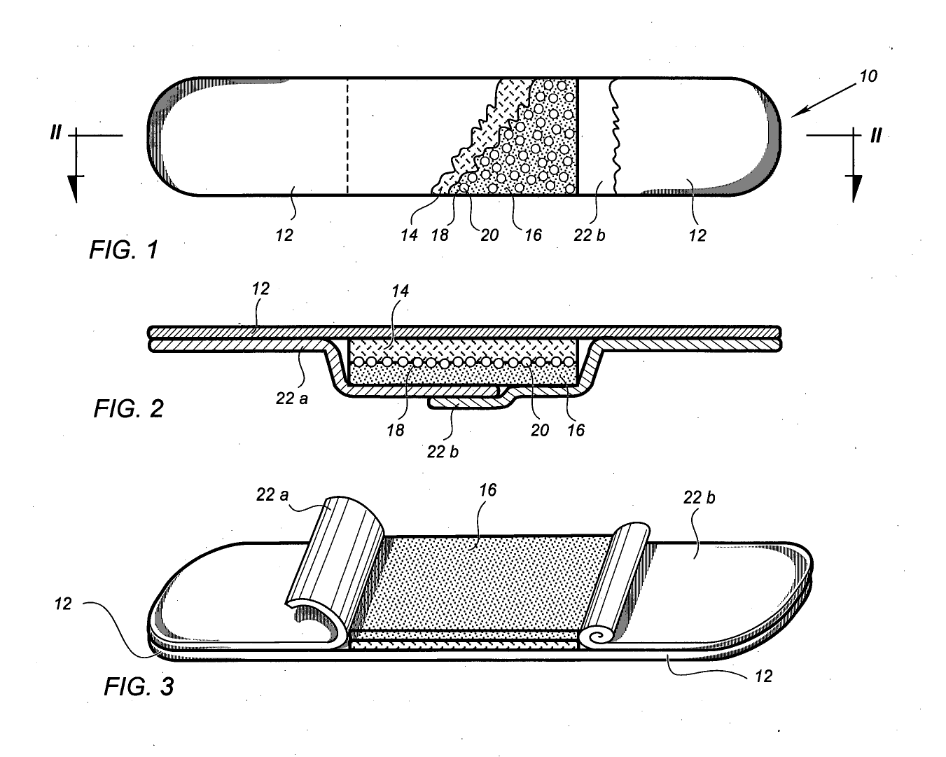

FIG. 1 is a top view of a plaster with one of the corners exposed to show the inner layers thereof according to a first preferred embodiment of the invention;

FIG. 2 is a section taken along line II-II of FIG. 1;

FIG. 3 is a bottom view of the plaster of FIG. 1;

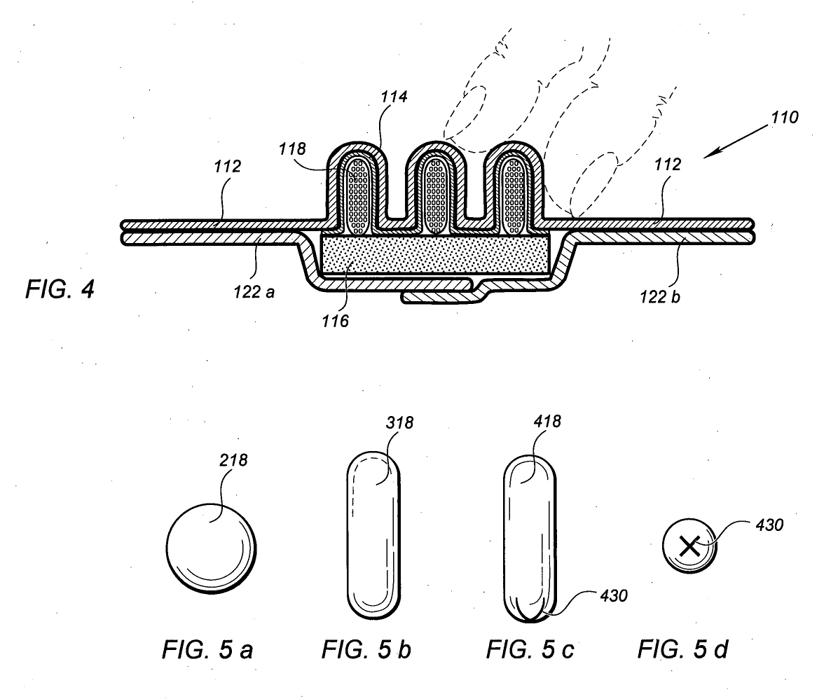

FIG. 4 is a cross-sectional view of a plaster according to a second embodiment of the present invention;

FIGS. 5a-5c illustrate optional forms of the medication containing capsules;

FIG. 5d is a bottom view of the capsule of FIG. 5c;

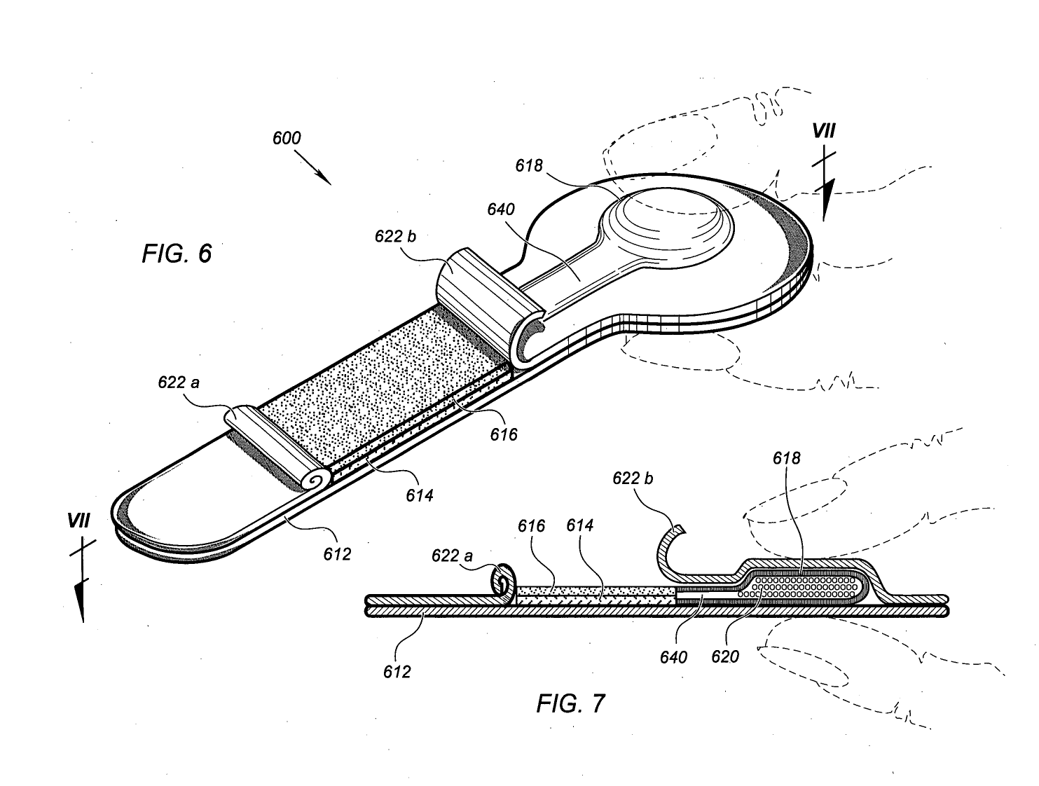

FIG. 6 is a perspective view of a plaster according to a third embodiment of the invention;

FIG. 7 is a section taken along line VII-VII of FIG. 6;

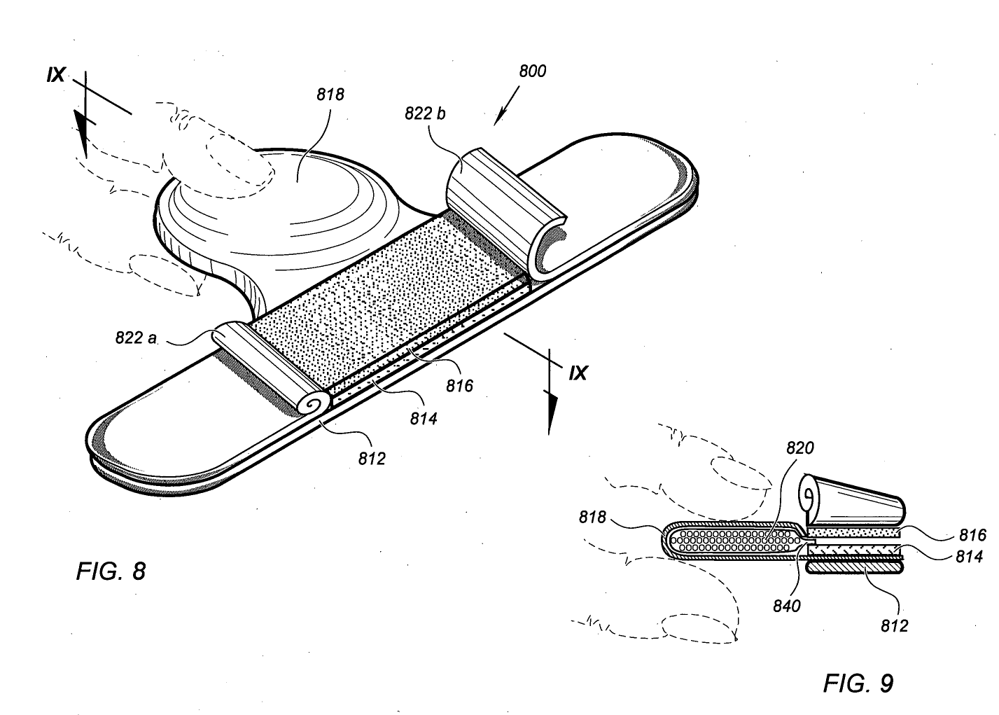

FIG. 8 is a perspective view of a plaster according to a fourth embodiment of the invention; and

FIG. 9 is a section taken along line IX-IX of FIG. 8.

### DETAILED DESCRIPTION OF THE PREFERRED EMBODIMENTS

In FIG. 1 there is shown a plaster generally denoted 10, which comprises a top adhesive layer 12, as conventionally known, an intermediate pad 14 and an external pad 16.

A pair of peal-off strips 22a and 22b are provided as in the conventional plasters (see FIGS. 2 and 3).

Capsules 18, containing a liquid or semi-liquid (such as ointment) medication substance 20 are interposed between intermediate pad 14 (shown upper in the drawing) and the external pad 16. Preferably, the capsules 18 are spherical (but see FIG. 5) and are made of a thin plastic material such as Polyethylene. Alternatively, an at-least partly permeable (“breathing”) foil may be used as sometimes requested by the drugs regulators.

Applying a slight pressure on the plaster will cause the rupture of the capsules 18 and the release of their content 20. The medication will be soaked in the pad 16 and penetrate through it to reach the wound.

Optionally, intermediate layer 14 is sterile, so that contact between medication 20 and pad 14 does not contaminate the medication. Optionally, pad 14 is made of an absorbing material. Preferably, the pad 14 is made out of a material different from that of pad 16. For example; in certain embodiments, pad 14 is made out of a less absorbing material than pad 16 to reduce or prevent wasting of the medication.

In some embodiments, pad 14 is made of a material which repels the medication. For example; in one such embodiment a pad with a hydrophobic surface facing the released medication is used with a hydrophilic medication. Such combination allows for minimizing medication waste.

The pad 16 is made of a sterile absorbing material and the cover 12 may be fabric, plastic or latex rubber, with or without an adhesive top layer.

It should be emphasized that the size of a capsule may vary from a few millimeters to approximately one centimeter. Optionally, one plaster may contain capsules of various sizes. The size may be determined according to the texture of the medication (whether it can be packaged at small volumes), the desired duration of treatment, the amount of medication needed, the size of the plaster and the nature of the wound.

The ability to place relatively large capsules in the plaster enables a less frequent replacing of the plaster in comparison to the conventional plaster. Also, when the capsules are relatively small at size, one could achieve a “slow-release” of the medication, if one presses the plaster in a way that only a certain part of the capsules burst each time, while others remain intact. This also contributes to less replacements of the plaster.

According to an additional unique feature of the invention, different medications may be packed into some of the capsules, depending on the injury type, and may be from the families of antibiotics, antiseptics, steroids, burn-healing medications, etc. A plaster containing several different types of capsules and/or containing different medications, will achieve a multi-task treatment never know before.

FIG. 4 illustrates a modified embodiment of the present invention in which the capsules 118 are elongated (“torpedo shaped”) and positioned upright. This position enables top access the capsules from outside the plaster, squeeze them to release the medication. In some embodiments, selective squeezing of one or more capsules, without squeezing other capsules provides a way to release only some of the medication, keeping the rest of the medication for use later, when other capsules have been exhausted.

Optionally, capsules, the contents of which can be released independently of each other are used for delivering medication to only a portion of pad 116. This may be useful, for instance, when pad 116 is larger than the wound, and the medication should not be applied to healthy skin.

FIGS. 5a-5c show various examples of the medication carrying capsule, namely in a spherical shape (FIG. 5a) and in “torpedo” shapes (FIGS. 5b-5c). Weakening lines 430 can be formed, as shown, in order to facilitate the bursting of the capsule upon pressure.

FIG. 6 shows another plaster, generally denoted 600, according to an embodiment of the invention. A section taken along line VII-VII of FIG. 6 is shown in FIG. 7. Plaster 600 is bulged at one side thereof for accommodating a squeezable medication container 618. Container 618 is adhered to adhesive cover 612. It further comprises a tubular neck 640, preferably integrally formed with container 618, for delivering medication from the container, preferably to an interface between intermediate pad 614 and external pad 616. The neck 640 should be as short as possible, to minimize the amount of medication that cannot be squeezed out of the container. A pair of such containers 618 can be provided, one at each side of the plaster 600.

FIG. 8 shows yet another embodiment of the invention. Plaster 800 is similar to plaster 600, but with the container 818 facing the pads along the longer edge of the product. Optionally, container 818 is not adhered to the skin next to the wound, and may be squeezed from both of its sides when the main portion of the plaster is adhered to the skin, without pressing the skin. A pair of such containers 818 can be provided, one at each side of the plaster 800.

Many changes, modifications, variations will become apparent to those skilled in the art after considering this specification and the accompanying drawings which disclose the preferred embodiments thereof. All such changes, modifications, and variations which do not depart from the spirit and scope of the invention are deemed to be covered by the invention, which is to be limited only by the claims which follow.

What is claimed is:

1. An adhesive plaster for treating a wounded area, the plaster comprising:
an adhesive cover;
a first pad of a liquid absorbent material for covering the wounded area;
at least one squeezable medication containing container, wherein the first pad and the container are arranged such that squeezing the container releases the medication to the first pad; and
a second pad adapted to separate released medication from the adhesive cover.

2. The plaster as claimed in claim 1, wherein at least one squeezable container comprises a capsule made of a material adapted to be ruptured under pressure applied thereagainst.

3. The plaster as claimed in claim 1, wherein the squeezable container has a narrowing portion, for releasing the medication when the container is squeezed, and preventing medication from being released when the container is not squeezed.

4. The plaster as claimed in any of the preceding claims, wherein at least a portion of said medication container is located between the first and the second pads.

5. The plaster as claimed in claim 3, wherein the narrowing portion, faces the first pad.

6. The plaster as claimed in any of the preceding claims, further comprising at-least one peal-off strip covering said first pad.

7. The plaster as claimed in claim 2 wherein the capsule is formed with weakening lines that facilitate its rupture upon a pressure applied thereto.

8. The plaster as claimed in claim 2 wherein the capsules contain two or more different medications to achieve a combined treatment.

9. The plaster as claimed in claim 2 wherein the capsules are made of an at-least partly permeable foil material.

10. The plaster as claimed in claim 9 wherein the capsules are made of Polyethylene.

11. The plaster as claimed in claim 10, wherein the capsules are spherical.

12. The plaster as claimed in claim 1, wherein the second pad extends between the first pad and the adhesive cover.

13. The plaster as claimed in claim 11, wherein said at least one container comprises a plurality of containers sandwiched between said first pad and said second pad.

14. The plaster as claimed in claim 1 or claim 2, wherein the containers are elongated and positioned upright in respect of the first pad.

15. The plaster as claimed in claim 14, wherein the elongated containers are spaced apart from each other so that each is accessible for being squeezed without squeezing the others.

16. The plaster as claimed in claim 1, comprising a squeezable container from which there extends an integrally formed elongated narrowing portion, the free end of the narrowing portion reaching between the first and second pads.

17. The plaster as claimed in claim 1, wherein said squeezable container is located at one end of an elongated side of the plaster.

18. The plaster as claimed in claim 1, wherein said squeezable container is located at one end of a transverse side of the plaster.
